(19) United States
(12) Patent Application Publication
(10) Pub. No.: US 2025/0051432 A1
(43) Pub. Date: Feb. 13, 2025

(54) METHODS OF TREATING CHRONIC GRAFT-VERSUS-HOST DISEASE USING AN ANTI-COLONY STIMULATING FACTOR 1 RECEPTOR ANTIBODY

(71) Applicant: Incyte Corporation, Wilmington, DE (US)

(72) Inventors: Chuan Tian, Glen Mills, PA (US); Shuyuan Lou, Wilmington, DE (US); Yan-ou Yang, Glen Mills, PA (US)

(21) Appl. No.: 18/778,310

(22) Filed: Jul. 19, 2024

Related U.S. Application Data
(60) Provisional application No. 63/607,809, filed on Dec. 8, 2023.
(60) Provisional application No. 63/528,398, filed on Jul. 23, 2023.

(51) Int. Cl. C07K 16/24; A61K 39/00; A61P 37/06

(52) U.S. Cl. CPC C07K 16/243; A61P 37/06; A61K 2039/505; A61K 2039/54; A61K 2039/545; A61K 2039/55; C07K 2317/565

(57) ABSTRACT

The present disclosure provides methods of treating chronic graft-versus-host disease (cGVHID) using an antibody that binds to colony stimulating factor 1 (CSF-1R).

### CLAIM OF PRIORITY

This application claims the benefit of U.S. Provisional Patent Application No. 63/528,398, filed on Jul. 23, 2023, and U.S. Provisional Patent Application No. 63/607,809, filed on Dec. 8, 2023, each of which is incorporated by reference herein in its entirety.

### SEQUENCE LISTING

This application contains a Sequence Listing that has been submitted electronically as an XML file named “20443-0823001_ST26_SL.XML.” The XML file, created on Jul. 12, 2024, is 13,789 bytes in size. The material in the XML file is hereby incorporated by reference in its entirety.

### BACKGROUND

Chronic graft-versus-host disease (cGVHD) is a severe complication of allogeneic hematopoietic cell transplantation (HCT) that affects various organs leading to a reduced quality of life. cGVHD occurs in up to 50% of allogeneic HCT cases, where donor T- and B-cells derived from the graft recognize and attack host antigens (Socié et al., 2014, Blood, 124:374-84). cGVHD has increased during the last two decades due to increasing patient age and increasing use of unrelated and/or mismatched donors, reduced intensity conditioning regimens, and peripheral blood as source for stem cells (Arai et al., 2015, Biol Blood Marrow Transplant, 21:266-74). Due to an increased risk of non-relapse mortality, cGVHD remains the leading cause for late mortality following allogeneic HCT (Zeiser et al., 2018, Blood, 131:1399-405; Li et al., 2019, Br J Haematol, 184:323-36).

### SUMMARY

Aspects of the present disclosure provide a method of treating chronic graft-versus-host disease (cGVHD) in a human subject in need thereof, the method comprising administering to the human subject a therapeutically effective amount of an antibody that binds to colony stimulating factor 1 receptor (CSF-1R), wherein the antibody comprises a variable heavy (VH) domain comprising VH complementarity determining region (CDR)1 (VH CDR1), VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a variable light (VL) domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11), and wherein (i) the human subject is an adult patient or a pediatric patient with a body weight greater than about 55 kg and the antibody is administered intravenously once every two weeks at a dose of about 22 mg; (ii) the human subject is a pediatric patient with a body weight greater than or equal to about 30 kg and less than or equal to about 55 kg and the antibody is administered intravenously once every two weeks at a dose of about 14 mg; or (iii) the human subject is a pediatric patient with a body weight less than about 30 kg and the antibody is administered intravenously once every two weeks at a dose of about 9 mg.

In some embodiments, the antibody is administered after failure of at least two prior lines of systemic therapy.

In some embodiments, the human subject is an adult patient or a pediatric patient with a body weight greater than about 55 kg and the antibody is administered intravenously once every two weeks at a dose of about 22 mg. In some embodiments, the human subject is an adult patient or a pediatric patient with a body weight greater than 55 kg and the antibody is administered intravenously once every two weeks at a dose of 22 mg. In some embodiments, the human subject is an adult patient or a pediatric patient with a body weight greater than about 55 kg and the antibody is administered intravenously once every two weeks at a dose of about 22 mg, wherein the antibody is administered after failure of at least two prior lines of systemic therapy. In some embodiments, the human subject is an adult patient or a pediatric patient with a body weight greater than 55 kg and the antibody is administered intravenously once every two weeks at a dose of 22 mg, wherein the antibody is administered after failure of at least two prior lines of systemic therapy.

In some embodiments, the human subject is a pediatric patient with a body weight greater than or equal to about 30 kg and less than or equal to about 55 kg and the antibody is administered intravenously once every two weeks at a dose of about 14 mg. In some embodiments, the human subject is a pediatric patient with a body weight greater than or equal to 30 kg and less than or equal to 55 kg and the antibody is administered intravenously once every two weeks at a dose of 14 mg. In some embodiments, the human subject is a pediatric patient with a body weight greater than or equal to about 30 kg and less than or equal to about 55 kg and the antibody is administered intravenously once every two weeks at a dose of about 14 mg, wherein the antibody is administered after failure of at least two prior lines of systemic therapy. In some embodiments, the human subject is a pediatric patient with a body weight greater than or equal to 30 kg and less than or equal to 55 kg and the antibody is administered intravenously once every two weeks at a dose of 14 mg, wherein the antibody is administered after failure of at least two prior lines of systemic therapy.

In some embodiments, the human subject is a pediatric patient with a body weight less than about 30 kg and the antibody is administered intravenously once every two weeks at a dose of about 9 mg. In some embodiments, the human subject is a pediatric patient with a body weight less than 30 kg and the antibody is administered intravenously once every two weeks at a dose of 9 mg. In some embodiments, the human subject is a pediatric patient with a body weight less than about 30 kg and the antibody is administered intravenously once every two weeks at a dose of about 9 mg, wherein the antibody is administered after failure of at least two prior lines of systemic therapy. In some embodiments, the human subject is a pediatric patient with a body weight less than 30 kg and the antibody is administered intravenously once every two weeks at a dose of 9 mg, wherein the antibody is administered after failure of at least two prior lines of systemic therapy.

In some embodiments, the human subject is an adult patient or a pediatric patient with a body weight greater than 55 kg and the antibody is administered intravenously once every two weeks at a dose of 22 mg; the human subject is a pediatric patient with a body weight greater than or equal to 30 kg and less than or equal to 55 kg and the antibody is administered intravenously once every two weeks at a dose of 14 mg; or the human subject is a pediatric patient with a body weight less than 30 kg and the antibody is administered intravenously once every two weeks at a dose of 9 mg.

In some embodiments, the human subject is an adult patient or a pediatric patient with a body weight greater than 55 kg and the antibody is administered intravenously once every two weeks at a dose of 22 mg; the human subject is a pediatric patient with a body weight greater than or equal to 30 kg and less than or equal to 55 kg and the antibody is administered intravenously once every two weeks at a dose of 14 mg; or the human subject is a pediatric patient with a body weight less than 30 kg and the antibody is administered intravenously once every two weeks at a dose of 9 mg, wherein the antibody is administered after failure of at least two prior lines of systemic therapy.

Aspects of the present disclosure provide a method of treating chronic graft-versus-host disease (cGVHD) in a human subject in need thereof, the method comprising administering to the human subject a therapeutically effective amount of an antibody that binds to colony stimulating factor 1 receptor (CSF-1R), wherein the antibody comprises a variable heavy (VH) domain comprising VH complementarity determining region (CDR)1 (VH CDR1), VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a variable light (VL) domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11), and wherein (i) the human subject has a body weight greater than about 60 kg and the antibody is administered intravenously once every two weeks at a dose of about 23 mg; (ii) the human subject has a body weight greater than or equal to about 40 kg and less than or equal to about 60 kg and the antibody is administered intravenously once every two weeks at a dose of about 17 mg; or (iii) the human subject has a body weight less than about 40 kg and the antibody is administered intravenously once every two weeks at a dose of about 10.5 mg.

In some embodiments, the antibody is administered after failure of at least two prior lines of systemic therapy.

In some embodiments, the human subject has a body weight greater than about 60 kg and the antibody is administered intravenously once every two weeks at a dose of about 23 mg. In some embodiments, the human subject has a body weight greater than 60 kg and the antibody is administered intravenously once every two weeks at a dose of 23 mg. In some embodiments, the human subject has a body weight greater than about 60 kg and the antibody is administered intravenously once every two weeks at a dose of about 23 mg, wherein the antibody is administered after failure of at least two prior lines of systemic therapy. In some embodiments, the human subject has a body weight greater than 60 kg and the antibody is administered intravenously once every two weeks at a dose of 23 mg, wherein the antibody is administered after failure of at least two prior lines of systemic therapy.

In some embodiments, the human subject has a body weight greater than or equal to about 40 kg and less than or equal to about 60 kg and the antibody is administered intravenously once every two weeks at a dose of about 17 mg. In some embodiments, the human subject has a body weight greater than or equal to 40 kg and less than or equal to about 60 kg and the antibody is administered intravenously once every two weeks at a dose of 17 mg. In some embodiments, the human subject has a body weight greater than or equal to about 40 kg and less than or equal to about 60 kg and the antibody is administered intravenously once every two weeks at a dose of about 17 mg, wherein the antibody is administered after failure of at least two prior lines of systemic therapy. In some embodiments, the human subject has a body weight greater than or equal to 40 kg and less than or equal to 60 kg and the antibody is administered intravenously once every two weeks at a dose of 17 mg, wherein the antibody is administered after failure of at least two prior lines of systemic therapy.

In some embodiments, the human subject has a body weight less than about 40 kg and the antibody is administered intravenously once every two weeks at a dose of about 10.5 mg. In some embodiments, the human subject has a body weight less than 40 kg and the antibody is administered intravenously once every two weeks at a dose of 10.5 mg. In some embodiments, the human subject has a body weight less than about 40 kg and the antibody is administered intravenously once every two weeks at a dose of about 10.5 mg, wherein the antibody is administered after failure of at least two prior lines of systemic therapy. In some embodiments, the human subject has a body weight less than 40 kg and the antibody is administered intravenously once every two weeks at a dose of 10.5 mg, wherein the antibody is administered after failure of at least two prior lines of systemic therapy.

In some embodiments, the human subject has a body weight greater than 60 kg and the antibody is administered intravenously once every two weeks at a dose of 23 mg; the human subject has a body weight greater than or equal to 40 kg and less than or equal to 60 kg and the antibody is administered intravenously once every two weeks at a dose of 17 mg; or the human subject has a body weight less than 40 kg and the antibody is administered intravenously once every two weeks at a dose of 10.5 mg.

In some embodiments, the human subject has a body weight greater than 60 kg and the antibody is administered intravenously once every two weeks at a dose of 23 mg; the human subject has a body weight greater than or equal to 40 kg and less than or equal to 60 kg and the antibody is administered intravenously once every two weeks at a dose of 17 mg; or the human subject has a body weight less than 40 kg and the antibody is administered intravenously once every two weeks at a dose of 10.5 mg, wherein the antibody is administered after failure of at least two prior lines of systemic therapy.

Aspects of the present disclosure provide a method of treating chronic graft-versus-host disease (cGVHD) in a pediatric human subject in need thereof, the method comprising administering to the pediatric human subject a therapeutically effective amount of an antibody that binds to colony stimulating factor 1 receptor (CSF-1R), wherein the antibody comprises a variable heavy (VH) domain comprising VH complementarity determining region (CDR)1 (VH CDR1), VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a variable light (VL) domain comprising VL CDR1, VL CDR2, and VL CDR3, wherein:
the VL CDR1 comprises the amino acid sequence LASEDIYDNLA (SEQ ID NO:9);
the VL CDR2 comprises the amino acid sequence YASSLQD (SEQ ID NO: 10); and
the VL CDR3 comprises the amino acid sequence LQDSEYPWT (SEQ ID NO:11), and
wherein the antibody is administered intravenously once every two weeks at a dose of 0.35 mg/kg body weight of the treated pediatric human subject.

In some embodiments, the cGVHD is recurrent or refractory active cGVHD. In some embodiments, the cGVHD is severe cGVHD.

In some embodiments, the human subject was prior treated with ibrutinib. In some embodiments, the human subject was prior treated with ruxolitinib.

In some embodiments, the human subject exhibited manifestations of cGVHD in greater than or equal to four organs at baseline. In some embodiments, the greater than or equal to four organs comprise the eyes. In some embodiments, the greater than or equal to four organs comprise the skin. In some embodiments, the greater than or equal to four organs comprise the eyes and the skin.

In some embodiments, the VH domain comprises the amino acid sequence EVTLKESGPALVKPTQTLTLTCTFSGFSLTTYGMGVGWIRQPPGKALEWLANIWWD DDKYYNPSLKNRLTISKDTSKNQVVLTMTNMDPVDTATYYCARIGPIKYPTAPYRYF DFWGQGTMVTVS (SEQ ID NO:4) and the VL domain comprises the amino acid sequence DIQMTQSPSSLSASVGDRVTITCLASEDIYDNLAWYQQKPGKAPKLLIYYASSLQDG VPSRFSGSGSGTDYTLTISSLQPEDFATYYCLQDSEYPWTFGGGTKVEIK (SEQ ID NO:5).

In some embodiments, the antibody comprises a heavy chain and a light chain, and wherein the heavy chain comprises the amino acid sequence set forth in SEQ ID NO:12 and the light chain comprises the amino acid sequence set forth in SEQ ID NO:3.

Aspects of the present disclosure provide a method of treating chronic graft-versus-host disease (cGVHD) in a population of human subjects in need thereof, the method comprising administering to each member of the population of human subjects a therapeutically effective amount of an antibody that binds to colony stimulating factor 1 receptor (CSF-1R), wherein the antibody comprises a variable heavy (VH) domain comprising VH complementarity determining region (CDR)1 (VH CDR1), VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a variable light (VL) domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11), wherein the antibody is administered intravenously once every two weeks at a dose of 0.3 mg/kg body weight of each treated human subject, and wherein the overall response rate in the population of human subjects within the first six months after initiation of treatment is at least 60%.

In some embodiments, the VH domain comprises the amino acid sequence EVTLKESGPALVKPTQTLTLTCTFSGFSLTTYGMGVGWIRQPPGKALEWLANIWWD DDKYYNPSLKNRLTISKDTSKNQVVLTMTNMDPVDTATYYCARIGPIKYPTAPYRYF DFWGQGTMVTVS (SEQ ID NO:4) and the VL domain comprises the amino acid sequence DIQMTQSPSSLSASVGDRVTITCLASEDIYDNLAWYQQKPGKAPKLLIYYASSLQDG VPSRFSGSGSGTDYTLTISSLQPEDFATYYCLQDSEYPWTFGGGTKVEIK (SEQ ID NO:5).

In some embodiments, the antibody comprises a heavy chain and a light chain, and wherein the heavy chain comprises the amino acid sequence set forth in SEQ ID NO:12 and the light chain comprises the amino acid sequence set forth in SEQ ID NO:3.

In some embodiments, the cGVHD is recurrent or refractory active cGVHD. In some embodiments, the cGVHD is recurrent or refractory active cGVHD after two or more lines of systemic therapy.

In some embodiments, the overall response rate in the population of human subjects within the first six months after initiation of treatment is at least 65%. In some embodiments, the overall response rate in the population of human subjects within the first six months after initiation of treatment is at least 70%. In some embodiments, the overall response rate in the population of human subjects within the first six months after initiation of treatment is at least 74%.

In some embodiments, the duration of response sensitivity in the human subject or the population of human subjects is at least 50%. In some embodiments, the duration of response sensitivity in the human subject or the population of human subjects is at least 55%. In some embodiments, the duration of response sensitivity in the human subject or the population of human subjects is at least 60%.

In some embodiments, the modified Lee Symptom Scale (mLSS) response rate in the population of human subjects is at least 45%. In some embodiments, the mLSS response rate in the population of human subjects is at least 50%. In some embodiments, the mLSS response rate in the population of human subjects is at least 55%.

In some embodiments, the antibody that binds to colony stimulating factor 1 receptor (CSF-1R) is axatilimab.

Other features and advantages of the invention will be apparent from the following detailed description, and from the claims.

### BRIEF DESCRIPTION OF THE DRAWINGS

FIG. 1 is a graph showing failure-free survival for patients treated with axatilimab at a dose of 0.3 mg/kg every two weeks.

FIG. 2 is a graph showing mLSS score for patients treated with axatilimab at a dose of 0.3 mg/kg every two weeks.

### DETAILED DESCRIPTION

The present disclosure provides methods of treating cGVHD in a human subject or a population of human subjects in need thereof by administering an antibody that binds to CSF-1R.

CSF-1R is a receptor for the cytokine colony stimulating factor 1 (CSF-1), which is responsible for the production, differentiation, and function of macrophages.

The amino acid sequence of the human CSF-1R protein is:

(SEQ ID NO: 1)
MGPGVLLLLLVATAWHGQGIPVIEPSVPELVVKPGATVTLRCVGNGSVEWDGPPSPHWTLYS
DGSSSILSTNNATFQNTGTYRCTEPGDPLGGSAAIHLYVKDPARPWNVLAQEVVVFEDQDAL
LPCLLTDPVLEAGVSLVRVRGRPLMRHTNYSFSPWHGFTIHRAKFIQSQDYQCSALMGGRKV
MSISIRLKVQKVIPGPPALTLVPAELVRIRGEAAQIVCSASSVDVNFDVFLQHNNTKLAIPQ
QSDFHNNRYQKVLTLNLDQVDFQHAGNYSCVASNVQGKHSTSMFFRVVESAYLNLSSEQNLI
QEVTVGEGLNLKVMVEAYPGLQGFNWTYLGPFSDHQPEPKLANATTKDTYRHTFTLSLPRLK
PSEAGRYSFLARNPGGWRALTFELTLRYPPEVSVIWTFINGSGTLLCAASGYPQPNVTWLQC
SGHTDRCDEAQVLQVWDDPYPEVLSQEPFHKVTVQSLLIVETLEHNQTYECRAHNSVGSGSW
AFIPISAGAHTHPPDEFLFTPVVVACMSIMALLLLLLLLLLYKYKQKPKYQVRWKIIESYEG
NSYTFIDPTQLPYNEKWEFPRNNLQFGKTLGAGAFGKVVEATAFGLGKEDAVLKVAVKMLKS
TAHADEKEALMSELKIMSHLGQHENIVNLLGACTHGGPVLVITEYCCYGDLLNFLRRKAEAM
LGPSLSPGQDPEGGVDYKNIHLEKKYVRRDSGFSSQGVDTYVEMRPVSTSSNDSFSEQDLDK
EDGRPLELRDLLHFSSQVAQGMAFLASKNCIHRDVAARNVLLINGHVAKIGDEGLARDIMND
SNYIVKGNARLPVKWMAPESIFDCVYTVQSDVWSYGILLWEIFSLGLNPYPGILVNSKFYKL
VKDGYQMAQPAFAPKNIYSIMQACWALEPTHRPTFQQICSFLQEQAQEDRRERDYTNLPSSS
RSGGSGSSSSELEEESSSEHLTCCEQGDIAQPLLQPNNYQFC.

Anti-CSF-1R antibodies can be used to treat cGVHD as described herein.

### Anti-Colony Stimulating Factor 1 Receptor Antibodies

The present disclosure provides methods of treating cGVHD by administering an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11).

In some embodiments, methods described herein comprise treating cGVHD by administering axatilimab.

Axatilimab (also known as SNDX-6352) is a humanized IgG4 monoclonal antibody that binds to CSF-1R and inhibits its function. Axatilimab is described in U.S. Pat. No. 9,908,939, which is incorporated by reference in its entirety.

The amino acid sequences of axatilimab heavy and light chains are shown below. Complementarity-determining regions (CDRs) 1, 2, and 3 of the variable heavy (VH) domain and the variable light (VL) domain are shown in that order from N-terminus to the C-terminus of the mature VL and VH sequences and are both underlined and boldened. With the exception of VHCDR-1, the CDRs were defined according to Kabat. The VHCDR-1 is defined according to the combined Kabat-Chothia definitions. Variable regions are underlined. An antibody comprising of the heavy chain (SEQ ID NO:2) and the light chain (SEQ ID NO:3) listed below is termed axatilimab.

### Axatilimab Heavy Chain:

(SEQ ID NO: 2)
EVTLKESGPALVKPTQTLTLTCTFSGFSLTTYGMGVGWIRQPPGKALEWLANIWWDDDKYYN
PSLKNRLTISKDTSKNQVVLTMTNMDPVDTATYYCARIGPIKYPTAPYRYFDFWGQGTMVTV
SSASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSRWQEGNVFSCSVM
HEALHNHYTQKSLSLSLGK

### Axatilimab Light Chain:

(SEQ ID NO: 3)
DIQMTQSPSSLSASVGDRVTITCLASEDIYDNLAWYQQKPGKAPKLLIYYASSLQDGVPSRF
SGSGSGTDYTLTISSLQPEDFATYYCLQDSEYPWTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC

The variable heavy (VH) domain of axatilimab has the following amino acid sequence:

(SEQ ID NO: 4)
EVTLKESGPALVKPTQTLTLTCTFSGFSLTTYGMGVGWIRQPPGKALEW
LANIWWDDDKYYNPSLKNRLTISKDTSKNQVVLTMTNMDPVDTATYYCA
RIGPIKYPTAPYRYFDFWGQGTMVTVS

The variable light (VL) domain of axatilimab has the following amino acid sequence:

(SEQ ID NO: 5)
DIQMTQSPSSLSASVGDRVTITCLASEDIYDNLAWYQQKPGKAPKLLIY
YASSLQDGVPSRFSGSGSGTDYTLTISSLQPEDFATYYCLQDSEYPWTF
GGGTKVEIK

The amino acid sequences of the VH CDRs of axatilimab are listed below:

VH CDR1:
(SEQ ID NO: 6)
GFSLTTYGMGVG;
VH CDR2:
(SEQ ID NO: 7)
NIWWDDDKYYNPSLKN;
and
VH CDR3:
(SEQ ID NO: 8)
IGPIKYPTAPYRYFDF.

The amino acid sequences of the VL CDRs of axatilimab are listed below:

VL CDR1:
(SEQ ID NO: 9)
LASEDIYDNLA;
VL CDR2:
(SEQ ID NO: 10)
YASSLQD;
and
VL CDR3:
(SEQ ID NO: 11)
LQDSEYPWT.

In certain embodiments, an anti-CSF-1R antibody described herein includes a human heavy chain and light chain constant region. In certain embodiments, the heavy chain constant region comprises a CH1 domain and a hinge region. In some embodiments, the heavy chain constant region comprises a CH2 domain. In some embodiments, the heavy chain constant region comprises a CH3 domain. In some embodiments, the heavy chain constant region comprises CH1, CH2 and CH3 domains. If the heavy chain constant region includes substitutions, such substitutions can modify the properties of the antibody (e.g., increase or decrease one or more of: Fc receptor binding, antibody glycosylation, the number of cysteine residues, effector cell function, or complement function). In certain embodiments, the antibody is an IgG antibody. In specific embodiments, the antibody is selected from the group consisting of IgG1, IgG2, IgG3, and IgG4.

In some embodiments, an anti-CSF-1R antibody is provided wherein a C-terminal residue of an antibody sequence described herein is cleaved, for example, the C-terminal residue of a heavy chain sequence, for example, a terminal lysine. Generally, the cleavage results from post-translation modifications of the expressed antibody. For example, an anti-CSF-1R antibody can include a heavy chain comprising the amino acid sequence set forth in SEQ ID NO:12 (below) and a light chain comprising the amino acid sequence set forth in SEQ ID NO:3.

(SEQ ID NO: 12)
EVTLKESGPALVKPTQTLTLTCTFSGFSLTTYGMGVGWIRQPPGKALEWLANIWWDDDKYYN
PSLKNRLTISKDTSKNQVVLTMTNMDPVDTATYYCARIGPIKYPTAPYRYFDFWGQGTMVTV
SSASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSRWQEGNVFSCSVM
HEALHNHYTQKSLSLSLG

Antibodies can be made, for example, by preparing and expressing synthetic genes that encode the recited amino acid sequences or by mutating human germline genes to provide a gene that encodes the recited amino acid sequences. Moreover, this antibody and other anti-CSF-1R antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16: 237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

The anti-CSF-1R antibodies can be in the form of full length antibodies, or in the form of low molecular weight forms (e.g., biologically active antibody fragments or minibodies) of the anti-CSF-1R antibodies, e.g., Fab, Fab′, F(ab′)₂, Fv, Fd, dAb, scFv, and sc(Fv)2. Other anti-CSF-1R antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an anti-CSF-1R antibody and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-CSF-1R antibody comprising at least one deamidation site, wherein the pH of the composition is from about 5.0 to about 6.5, such that, e.g., at least about 90% of the anti-CSF-1R antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.0, 5.1, 5.2, 5.3, 5.4, 5.5, 5.6, 5.7, 5.8, 5.9, 6.0, 6.1, 6.2, 6.3, 6.4 or 6.5.

An “acidic variant” is a variant of a polypeptide of interest which is more acidic (e.g., as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e., the neutral amide side chain has been converted to a residue with an overall acidic character.

The term “mixture” as used herein in reference to a composition comprising an anti-CSF-1R antibody means the presence of both the desired anti-CSF-1R antibody and one or more acidic variants thereof. The acidic variants may comprise predominantly deamidated anti-CSF-1R antibody, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 1%, 50%, 3%, 2% or 1%.

### Bispecific Antibodies

In certain embodiments, an anti-CSF-1R antibody described herein (e.g., axatilimab or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11)) is present in a bispecific antibody.

Bispecific antibodies are antibodies that have binding specificities for at least two different epitopes. Exemplary bispecific antibodies may bind to two different epitopes of the CSF-1R protein. Other such antibodies may combine a CSF-1R binding site with a binding site for another protein. Bispecific antibodies can be prepared as full length antibodies or low molecular weight forms thereof (e.g., F(ab′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH₃ domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

In certain embodiments, an anti-CSF-1R antibody thereof described herein (e.g., axatilimab or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11)) is present in a multivalent antibody.

A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

The anti-CSF-1R antibodies disclosed herein (e.g., axatilimab or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11)) may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g., ⁹⁰Y, ¹³¹1) fluorescent substances, luminescent substances, haptens, enzymes, metal chelates, drugs, and toxins (e.g., calicheamicin, Pseudomonas exotoxin A, ricin (e.g., deglycosylated ricin A chain), exatecan, auristatins (e.g., auristatin E), maytansine, pyrrolobenzodiazepine (PBD)).

In one embodiment, to improve the cytotoxic actions of anti-CSF-1R antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-CSF-1R antibody is modified with a moiety that improves its stabilization and/or retention in circulation, e.g., in blood, serum, or other tissues, e.g., by at least about 1.5, 2, 5, 10, or 50 fold. For example, the anti-CSF-1R antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-CSF-1R antibody can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 5,156,840).

### Methods of Producing Antibodies

Antibodies may be produced in, for example, bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5a, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-CSF-1R antibody (e.g., axatilimab) is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Antibody Pharmaceutical Compositions and Administration

An anti-CSF-1R antibody described herein (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) can be formulated as a pharmaceutical composition for administration to a human subject, e.g., to treat a disorder described herein.

Typically, a pharmaceutical composition includes a pharmaceutically acceptable carrier. As used herein, pharmaceutically acceptable carrier includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X). Suitable anti-CSF-1R antibody formulations are described, e.g., in U.S. Pat. No. 10,039,826 B2, which is incorporated by reference in its entirety.

The anti-CSF-1R antibody can be administered to a human subject or a population of human subjects having cGVHD. In some embodiments, the anti-CSF-1R antibody can be administered to a human subject or a population of human subjects having recurrent or refractory active cGVHD. In some embodiments, the anti-CSF-1R antibody can be administered to a human subject or a population of human subjects having recurrent or refractory active cGVHD after two or more lines of systemic therapy (e.g., ruxolitinib, belumosudil, ibrutinib).

The anti-CSF-1R antibody can be administered to a human subject or a population of human subjects having cGVHD who have received one or more previous cGVHD treatments. In some embodiments, the human subject or the population of human subjects has received at least two previous cGVHD treatments. In some embodiments, the human subject or the population of human subjects has received at least three previous cGVHD treatments. In some embodiments, the human subject or the population of human subjects has received at least four previous cGVHD treatments. In some embodiments, the human subject or the population of human subjects has received at least five previous cGVHD treatments. In some embodiments, the human subject or the population of human subjects has received at least six previous cGVHD treatments.

The anti-CSF-1R antibody can be administered to a human subject after failure of at least two prior lines of systemic therapy, which can include one or more of ruxolitinib, belumosudil, and ibrutinib. In some embodiments, the human subject was prior treated with ibrutinib. In some embodiments, the human subject was prior treated with ruxolitinib. In some embodiments, the human subject was prior treated with belumosudil.

The anti-CSF-1R antibody can be administered to a human subject or each member of a population of human subjects, e.g., each member of a population of human subjects in need thereof, for example, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody can also be tailored for a human subject or each member of a population of human subjects, e.g., by monitoring the human subject, e.g., using tomographic imaging, e.g., to visualize an organ.

The antibody can be administered in a mg/kg subject weight dose (as used herein, “mg/kg” refers to milligrams of an antibody administered per kilogram of body weight of the treated subject).

A human subject to be treated according to methods described herein can be an adult patient or a pediatric patient. As used herein, “an adult patient” refers to a human subject that is 18 years of age or older. As used herein, “a pediatric patient” or “a pediatric human subject” refers to a human subject that is under 18 years of age. In some embodiments, a pediatric patient or a pediatric human subject can include a human subject who is 6 years of age or older. In some embodiments, a pediatric patient or a pediatric human subject can include a human subject who is aged from birth through the first 28 days of life; from 29 days to less than 2 years old; from 2 years to less than 12 years old; or from 12 years to 17 years old.

An exemplary weight-based dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) to a human subject (e.g., an adult patient) at a dosage of about 0.3 mg/kg once every two weeks.

As used herein, “about” when referring to a measurable value such as an amount, a dosage, a temporal duration, and the like, is meant to encompass variations of 10%. For example, with respect to doses or dosages, the term “about” denotes a range that is +10% of a recited dose, such that, for example, a dose of about 0.3 mg/kg will be between 0.27 mg/kg and 0.33 mg/kg subject weight. Exemplary doses included within “about 0.3 mg/kg” are 0.27 mg/kg, 0.28 mg/kg, 0.29 mg/kg, 0.3 mg/kg, 0.31 mg/kg, 0.32 mg/kg, and 0.33 mg/kg. Exemplary doses included within “about 0.35 mg/kg” are 0.32 mg/kg, 0.33 mg/kg, 0.34 mg/kg, 0.35 mg/kg, 0.36 mg/kg, 0.37 mg/kg, and 0.38 mg/kg. In another example, a body weight of “about 55 kg” encompasses 49.5 kg to 60.5 kg and a dose of “about 22 mg” encompasses 19.8 mg to 24.2 mg. Exemplary body weights included within “about 55 kg” are 50 kg, 51 kg, 52 kg, 53 kg, 54 kg, 55 kg, 56 kg, 57 kg, 58 kg, 59 kg, and 60 kg. Exemplary doses included within “about 22 mg” are 20 mg, 21 mg, 22 mg, 23 mg, and 24 mg.

A further exemplary weight-based dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) to a human subject (e.g., an adult patient) at a dosage of 0.3 mg/kg once every two weeks.

An exemplary weight-based dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) to a human subject (e.g., pediatric patient) at a dosage of about 0.35 mg/kg once every two weeks.

A further exemplary weight-based dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) to a human subject (e.g., pediatric patient) at a dosage of 0.35 mg/kg once every two weeks.

The antibody can be administered in a flat dose (as used herein, “flat dose” refers to milligrams of an antibody administered to all treated subjects regardless of their weight, age or other factors).

An exemplary flat dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) to a human subject at a dosage of about 22 mg once every two weeks.

A further exemplary flat dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) to a human subject at a dosage of 22 mg once every two weeks.

The antibody can be administered in a weight-band dose. As used herein, “weight-band dose” refers to milligrams of an antibody administered per pre-defined ranges (or bands) of body weight of the treated subject.

An exemplary weight-band dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) at a dosage of (i) about 22 mg once every two weeks to an adult patient or a pediatric patient with a body weight greater than about 55 kg; (ii) about 14 mg once every two weeks to a pediatric patient with a body weight greater than or equal to about 30 kg and less than or equal to about 55 kg; or (iii) about 9 mg once every two weeks to a pediatric patient with a body weight less than about 30 kg. In some embodiments, the pediatric patient is 6 years of age or older.

A further exemplary weight-band dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab), after failure of at least two prior lines of systemic therapy, at a dosage of (i) about 22 mg once every two weeks to an adult patient or a pediatric patient with a body weight greater than about 55 kg; (ii) about 14 mg once every two weeks to a pediatric patient with a body weight greater than or equal to about 30 kg and less than or equal to about 55 kg; or (iii) about 9 mg once every two weeks to a pediatric patient with a body weight less than about 30 kg. In some embodiments, the pediatric patient is 6 years of age or older.

A further exemplary weight-band dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) at a dosage of (i) 22 mg once every two weeks to an adult patient or a pediatric patient with a body weight greater than 55 kg; (ii) 14 mg once every two weeks to a pediatric patient with a body weight greater than or equal to about 30 kg and less than or equal to 55 kg; or (iii) 9 mg once every two weeks to a pediatric patient with a body weight less than 30 kg. In some embodiments, the pediatric patient is 6 years of age or older.

A further exemplary weight-band dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab), after failure of at least two prior lines of systemic therapy, at a dosage of (i) 22 mg once every two weeks to an adult patient or a pediatric patient with a body weight greater than 55 kg; (ii) 14 mg once every two weeks to a pediatric patient with a body weight greater than or equal to about 30 kg and less than or equal to 55 kg; or (iii) 9 mg once every two weeks to a pediatric patient with a body weight less than 30 kg. In some embodiments, the pediatric patient is 6 years of age or older.

An exemplary weight-band dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) at a dosage of (i) about 23 mg once every two weeks to a human subject with a body weight greater than about 60 kg; (ii) about 17 mg once every two weeks to a human subject with a body weight greater than or equal to about 40 kg and less than or equal to about 60 kg; or (iii) about 10.5 mg once every two weeks to a human subject with a body weight less than about kg. In some embodiments, the human subject is an adult patient. In some embodiments, the human subject is a pediatric patient. In some embodiments, the pediatric patient is 6 years of age or older.

A further exemplary weight-band dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab), after failure of at least two prior lines of systemic therapy, at a dosage of (i) about 23 mg once every two weeks to a human subject with a body weight greater than about 60 kg; (ii) about 17 mg once every two weeks to a human subject with a body weight greater than or equal to about 40 kg and less than or equal to about 60 kg; or (iii) about 10.5 mg once every two weeks to a human subject with a body weight less than about 40 kg. In some embodiments, the human subject is an adult patient. In some embodiments, the human subject is a pediatric patient. In some embodiments, the pediatric patient is 6 years of age or older.

A further exemplary weight-band dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) at a dosage of (i) 23 mg once every two weeks to a human subject with a body weight greater than 60 kg; (ii) 17 mg once every two weeks to a human subject with a body weight greater than or equal to 40 kg and less than or equal to 60 kg; or (iii) 10.5 mg once every two weeks to a human subject with a body weight less than 40 kg. In some embodiments, the human subject is an adult patient. In some embodiments, the human subject is a pediatric patient. In some embodiments, the pediatric patient is 6 years of age or older.

A further exemplary weight-band dosing regimen comprises intravenous administration of an anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab), after failure of at least two prior lines of systemic therapy, at a dosage of (i) 23 mg once every two weeks to a human subject with a body weight greater than 60 kg; (ii) 17 mg once every two weeks to a human subject with a body weight greater than or equal to 40 kg and less than or equal to 60 kg; or (iii) 10.5 mg once every two weeks to a human subject with a body weight less than 40 kg. In some embodiments, the human subject is an adult patient. In some embodiments, the human subject is a pediatric patient. In some embodiments, the pediatric patient is 6 years of age or older.

An anti-CSF-1R antibody (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) dose can be administered biweekly (every two weeks).

A pharmaceutical composition may include a therapeutically effective amount of an anti-CSF-1R antibody described herein (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab).

The term “therapeutically effective amount of an agent is that amount sufficient to effect beneficial or desired results, for example, clinical results, and, as such, a “therapeutically effective amount” depends upon the context in which it is being applied. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

In some embodiments, methods described herein comprise administering, to each member of a population of human subjects, a therapeutically effective amount of an anti-CSF-1R antibody described herein (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab), wherein the antibody is administered intravenously once every two weeks at a dose of 0.3 mg/kg body weight of each treated human subject, and wherein the overall response rate in the population of human subjects within the first six months after initiation of treatment is at least 60% (e.g., at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, or at least 80%). In such instances, the overall response rate is the proportion of patients in each dose group who achieved an objective response (either a complete response (CR) or a partial response (PR)) as defined by 2014 NIH Consensus Criteria for cGVHD by cycle 7 (28-day cycles), day 1 (C7D1). See Example below.

In some embodiments, methods described herein comprise administering, to a human subject or to each member of a population of human subjects, a therapeutically effective amount of an anti-CSF-1R antibody described herein (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO: 10); and the VL CDR3 comprises the amino acid sequence LQDSEYPWT (SEQ ID NO:11) or axatilimab), wherein the antibody is administered intravenously once every two weeks at a dose of 0.3 mg/kg body weight of each treated human subject, and wherein the duration of response sensitivity in the human subject or the population of human subjects is at least 50% (e.g., at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, or at least 70%). In such instances, the duration of response sensitivity is defined as the interval from the date of initial overall response of CR or PR until (i) documented progression of cGVHD as defined by the NIH Consensus Development Project on Clinical trials in cGVHD (Lee et al., 2015), or start of new anti-GVHD systemic therapy, or death from any cause, whichever is earlier; and (ii) alternative measure of durability of response: start of new anti-GVHD systemic therapy, or death from any cause, whichever is earlier. See Example below.

In some embodiments, methods described herein comprise administering, to each member of a population of human subjects, a therapeutically effective amount of an anti-CSF-1R antibody described herein (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab), wherein the antibody is administered intravenously once every two weeks at a dose of 0.3 mg/kg body weight of each treated human subject, and wherein the modified Lee Symptom Scale (mLSS) response rate in the population of human subjects is at least 45% (e.g., at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, or at least 65%). In such instances, the mLSS response rate is the duration of ≥7 point improvement in mLSS defined as the summation of the time in the post-baseline cycles for which the decrease in the total score from baseline is ≥7 points, the summation is calculated as the sum of anytime in post-baseline cycles with a ≥7 points decreasing in the total score compared to baseline. See Example below.

### Indications

An anti-CSF-1R antibody described herein (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO:11) or axatilimab) can be used to treat chronic graft-versus-host disease (cGVHD) in a human subject or a population of human subjects in need thereof. In some embodiments, the human subject is an adult patient. In some embodiments, the human subject is a pediatric patient. In some embodiments, the pediatric patient is 6 years of age or older. In some embodiments, the human subject was prior treated with ibrutinib, ruxolitinib, or ibrutinib and ruxolitinib. In some embodiments, the human subject was prior treated with ibrutinib, ruxolitinib, belumosudil, or a combination of any of these. In some embodiments, the human subject exhibited manifestations of cGVHD in greater than or equal to four organs at baseline. In some embodiments, the greater than or equal to four organs at baseline are selected from mouth, esophagus, small intestine, colon, liver, joints and fascia, eyes, skin, and lungs. In some embodiments, the greater than or equal to four organs at baseline comprise the eyes, the skin, or the eyes and the skin. In some embodiments, the cGVHD is recurrent or refractory active cGVHD. In some embodiments, the cGVHD is severe cGVHD. In some embodiments, the cGVHD is recurrent or refractory active cGVHD after two or more lines of systemic therapy. In some embodiments, the cGVHD is recurrent or refractory active cGVHD after three or more lines of systemic therapy. In some embodiments, the cGVHD is recurrent or refractory active cGVHD after four or more lines of systemic therapy. In some embodiments, the cGVHD is recurrent or refractory active cGVHD after five or more lines of systemic therapy. In some embodiments, the cGVHD is recurrent or refractory active cGVHD after six or more lines of systemic therapy.

Another aspect comprises an anti-CSF-1R antibody described herein (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO: 11) or axatilimab) for use in the treatment of cGVHD in a human subject or a population of human subjects in need thereof. In some embodiments, the human subject is an adult patient. In some embodiments, the human subject is a pediatric patient. In some embodiments, the pediatric patient is 6 years of age or older. In some embodiments, the human subject was prior treated with ibrutinib, ruxolitinib, or ibrutinib and ruxolitinib. In some embodiments, the human subject was prior treated with ibrutinib, ruxolitinib, belumosudil, or a combination of any of these. In some embodiments, the human subject exhibited manifestations of cGVHD in greater than or equal to four organs at baseline. In some embodiments, the greater than or equal to four organs at baseline are selected from mouth, esophagus, small intestine, colon, liver, joints and fascia, eyes, skin, and lungs. In some embodiments, the greater than or equal to four organs at baseline comprise the eyes, the skin, or the eyes and the skin. In some embodiments, the cGVHD is recurrent or refractory active cGVHD. In some embodiments, the cGVHD is severe cGVHD. In some embodiments, the cGVHD is recurrent or refractory active cGVHD after two or more lines of systemic therapy. In some embodiments, the cGVHD is recurrent or refractory active cGVHD after three or more lines of systemic therapy. In some embodiments, the cGVHD is recurrent or refractory active cGVHD after four or more lines of systemic therapy. In some embodiments, the cGVHD is recurrent or refractory active cGVHD after five or more lines of systemic therapy. In some embodiments, the cGVHD is recurrent or refractory active cGVHD after six or more lines of systemic therapy.

Another aspect comprises an anti-CSF-1R antibody described herein (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a VL domain comprising VL CDR1, VL CDR2, and VL CDR3, wherein the VL CDR1 comprises the amino acid sequence LASEDIYDNLA (SEQ ID NO:9); the VL CDR2 comprises the amino acid sequence YASSLQD (SEQ ID NO:10); and the VL CDR3 comprises the amino acid sequence LQDSEYPWT (SEQ ID NO: 11) or axatilimab) in the manufacture of a medicament for treating cGVHD in a human subject or a population of human subjects in need thereof. In some embodiments, the human subject is an adult patient. In some embodiments, the human subject is a pediatric patient. In some embodiments, the pediatric patient is 6 years of age or older. In some embodiments, the human subject was prior treated with ibrutinib, ruxolitinib, or ibrutinib and ruxolitinib. In some embodiments, the human subject was prior treated with ibrutinib, ruxolitinib, belumosudil, or a combination of any of these. In some embodiments, the human subject exhibited manifestations of cGVHD in greater than or equal to four organs at baseline. In some embodiments, the greater than or equal to four organs at baseline are selected from mouth, esophagus, small intestine, colon, liver, joints and fascia, eyes, skin, and lungs. In some embodiments, the greater than or equal to four organs at baseline comprise the eyes, the skin, or the eyes and the skin. In some embodiments, the treated subject has advanced cGVHD. In some embodiments, the cGVHD is recurrent or refractory active cGVHD. In some embodiments, the cGVHD is severe cGVHD. In some embodiments, the cGVHD is recurrent or refractory active cGVHD after two or more lines of systemic therapy. In some embodiments, the cGVHD is recurrent or refractory active cGVHD after three or more lines of systemic therapy. In some embodiments, the cGVHD is recurrent or refractory active cGVHD after four or more lines of systemic therapy. In some embodiments, the cGVHD is recurrent or refractory active cGVHD after five or more lines of systemic therapy. In some embodiments, the cGVHD is recurrent or refractory active cGVHD after six or more lines of systemic therapy.

In some embodiments, the treated subject has received at least two previous cGVHD treatments. In some embodiments, the treated subject has received at least three previous cGVHD treatments. In some embodiments, the treated subject has received at least four previous cGVHD treatments. In some embodiments, the treated subject has received at least five previous cGVHD treatments. In some embodiments, the treated subject has received at least six previous cGVHD treatments. In some embodiments, the treated subject has received greater than four previous cGVHD treatments. In some embodiments, the treated subject has received between four and six previous cGVHD treatments. In some embodiments, the treated subject has received greater than six previous cGVHD treatments. In some embodiments, the human subject was prior treated with ibrutinib, ruxolitinib, or ibrutinib and ruxolitinib.

In some embodiments, the human subject exhibited manifestations of cGVHD in greater than or equal to four organs at baseline. In some embodiments, the greater than or equal to four organs at baseline are selected from mouth, esophagus, small intestine, colon, liver, joints and fascia, eyes, skin, and lungs. In some embodiments, the greater than or equal to four organs at baseline comprise the eyes, the skin, or the eyes and the skin.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Efficacy, Safety, and Tolerability of Axatilimab in Advanced Chronic Graft-Versus-Host Disease

Efficacy, safety, and tolerability of axatilimab was assessed in a randomized phase 2 study involving a total of 241 adult and pediatric patients with recurrent or refractory active cGVHD whose disease had progressed after two or more prior therapies. Patients enrolled in this study received a median of four prior systemic therapies. The number of patients enrolled in the study that failed treatment with ruxolitinib, belumosudil, and ibrutinib was 71%, 20%, and 34%, respectively. Fifty-six percent (56%) of patients had manifestations of cGVHD in greater than or equal to four organs at baseline. See Table 1 below.

TABLE 1
Patient population

| | Total cohort |
|---|---|
| Patient characteristic | (N = 241) |
| Age, median (min, max), y | 53 (7, 81) |
| Sex, male, n (%) | 151 (62.7) |
| Race, White, n (%) | 200 (83.0) |
| Time from cGVHD diagnosis to randomization, | 4.01 (0.4, 17.6) |
| median (min, max), y | |
| Number of prior systemic cGVHD therapy, | 4 (2, 15) |
| median (min, max) | |
| Prior systemic cGVHD therapy^{a}, n (%) | 204 (84.6) |
| Prior ibrutinib, n (%) | 75 (31.1) |
| Prior ruxolitinib, n (%) | 179 (74.3) |
| Prior belumosudil, n (%) | 56 (23.2) |
| Number of organs involved at baseline, median | 4 (0, 8) ^{b} |
| (min, max) | |
| ≥4 organs involved, n (%) | 130 (53.9) |
| Patients with severe disease, n (%) | 192 (79.7) |

^{a}Prior use of at least 1 of the following therapies: ibrutinib, ruxolitinib, or belumosudil.
^{b}No patients on study had 0 organs involved. 2 patients consented but withdrew from study before the first dosing visit, in which organ involvement information was collected.

In this study, patients were randomized into one of three treatment groups:
0.3 mg/kg every two weeks [n=80];
1 mg/kg every two weeks [n=81]; or
3 mg/kg every four weeks [n=80].

The study's primary endpoint was the overall response (OR) rate, which is the proportion of patients in each dose group who achieved an objective response (either a complete response (CR) or a partial response (PR)) as defined by 2014 NIH Consensus Criteria for cGVHD by cycle 7 (28-day cycles), day 1 (C7D1).

The study's secondary endpoints include duration of response (DOR) and validated quality-of-life assessments using the Modified Lee Symptom Scale (mLSS). Evaluation of mLSS was performed according to Lee et al., Development and validation of a scale to measure symptoms of chronic graft-versus-host disease. Biol Blood Marrow Transplant (2002) 8(8): 444-452; and Teh et al., Reliability and Validity of the Modified 7-Day Lee Chronic Graft-versus-Host Disease Symptom Scale. Biol Blood Marrow Transplant (2020) 26(3): 562-567. Duration of response was calculated using the following criteria:
DOR sensitivity: Sensitivity analysis was performed and the DOR defined as the interval from the date of initial overall response of CR or PR until:
   Documented progression of cGVHD as defined by the NIH Consensus Development Project on Clinical trials in cGVHD (Lee et al., 2015), or start of new anti-GVHD systemic therapy, or death from any cause, whichever is earlier.
   Alternative measure of durability of response: start of new anti-GVHD systemic therapy, or death from any cause, whichever is earlier. Patients who haven't started new therapy and still alive will be censored at the last contact date. Similar method as primary analysis of DOR was applied.
DOR FDA definition: DOR (days) defined as the interval from the date of initial response of CR or PR until the first progression of cGVHD from nadir in any organ or start of new anti-GVHD systemic therapy, or death from any cause, whichever is earlier.
DOR protocol definition: DOR defined as the time from initial response (including partial response (PR) or complete response (CR)) until documented progression of cGVHD, start of new therapy, or death for any reason.

Time to event variable DOR was summarized descriptively using the Kaplan-Meier method for each dose cohort for each definition separately in patients who are responders. Point estimates and 2-sided 95% Cis for median using the Brookmeyer-Crowley (1982) method was estimated. Plots of the Kaplan-Meier estimate for the duration of response are presented by dose cohort. Variable DOR (days) is defined as the interval from the date of initial response of CR or PR until the first progression of cGVHD from nadir in any organ or start of new anti-GVHD systemic therapy, or death from any cause, whichever is earlier

For each DOR definition, the sustained response rate (SRR) was also calculated. SRR is defined as the proportion of patients with sustained response. The sustained response is defined as OR lasting for at least 20 weeks (140 days) from the time of initial response.

Responses by organ system will be assessed based on the 2014 NIH Consensus Development Project on Clinical Trials in cGVHD (see Lee et al., Measuring therapeutic response in chronic graft-versus-host disease. National Institutes of Health consensus development project on criteria for clinical trials in chronic graft-versus-host disease: IV. The 2014 Response Criteria Working Group report. Biol Blood Marrow Transplant (2015) 21(6): 984-999). CR is defined as resolution of all manifestations in each organ or site, and PR is defined as improvement in at least 1 organ or site without progression in any other organ or site.

### Results

The clinical trial results described below demonstrate that axatilimab was well tolerated and resulted in robust clinical activity and durable responses.

### Primary Endpoints

Surprisingly, patients treated with axatilimab at the lowest dose (0.3 mg/kg Q2W) demonstrated higher overall response rates compared to patients treated with the higher doses of axatilimab (1.0 mg/kg Q2W; 3.0 mg/kg Q4W).

As shown in Table 2, patients treated with axatilimab at doses of 0.3 mg/kg every two weeks, 1.0 mg/kg every two weeks, and 3.0 mg/kg every four weeks demonstrated overall response rates within the first six months of treatment of 74%, 67%, and 50%, respectively (95% Confidence Interval).

TABLE 2
Overall response rates per treatment group

| | 0.3 mg/kg | 1 mg/kg | 3 mg/kg |
|---|---|---|---|
| | Q2W | Q2W | Q4W |
| Parameter | (N = 80) | (N = 81) | (N = 80) |
| OR within C7D1, n(%) | 59 (74) | 54 (67) | 40 (50) |
| 95% CI (%) | (63, 83) | (55, 77) | (39, 61) |
| CR, n(%) | 1 (1.3) | 0 | 1 (1.3) |
| PR, n(%) | 58 (72) | 54 (67) | 39 (49) |

It was also surprisingly shown that 60% of patients treated with axatilimab at the lowest dose (0.3 mg/kg Q2W) maintained a response at 12 months, based on Kaplan Meir estimate, while only 53% of patients treated with axatilimab at the highest dose (3.0 mg/kg Q4W) maintained a response at 12 months. The number of patients treated with axatilimab at the intermediate dose (1.0 mg/kg Q2W) who maintained a response at 12 months was similar to that of patients treated with axatilimab at the lowest dose (0.3 mg/kg Q2W). Similar results were observed when DOR was calculated using the FDA definition and the protocol definition as well as when DOR included SRR. See Table 3 below.

TABLE 3
Duration of response rates per treatment group

| | 0.3 mg/kg | 1 mg/kg | 3 mg/kg |
|---|---|---|---|
| | Q2W | Q2W | Q4W |
| Parameter | (N = 80) | (N = 81) | (N = 80) |
| DOR Sensitivity (12 month | 60 (43, 74) | 60 (43, 74) | 53 (30, 71) |
| event-free KM estimate), % | | | |
| DOR FDA definition (months) | 1.9 (1.2, 3.7) | 2.2 (1.4, 5.6) | 2.3 (1.1, 4.9) |
| DOR Protocol definition (months) | 7.1 (2.0, NE) | 5.5 (3.6, 6.9) | 4.9 (2.8, 9.5) |
| DOR Sensitivity SRR, n(%) | 40 (50) | 40 (49) | 30 (38) |
| DOR FDA SRR, n(%) | 17 (21) | 14 (17) | 11 (14) |
| DOR Protocol definition SRR, n(%) | 28 (35) | 22 (27) | 18 (22) |

As shown in Table 4, the time to response for patients treated with axatilimab at doses of 0.3 mg/kg Q2W, 1.0 mg/kg Q2W, and 3.0 mg/kg Q4W was 1.7 months, 1.9 months, and 1.4 months, respectively.

TABLE 4
Time to response per treatment group

| | 0.3 mg/kg | 1 mg/kg | 3 mg/kg |
|---|---|---|---|
| | Q2W | Q2W | Q4W |
| Parameter | (N = 80) | (N = 81) | (N = 80) |
| Time to response, | 1.7 (0.9-8.1) | 1.9 (0.9-8.6) | 1.4 (0.9-5.6) |
| median months (range) | | | |

Responses Across Patient Subgroups in the Lowest Dose Treatment Group (0.3 mg/kg Q2W)

Responses for patients treated with axatilimab at the lowest dose (0.3 mg/kg Q2W) were achieved across key patient subgroups, including those with prior exposure to ruxolitinib, belumosudil, and/or ibrutinib. Greater than or equal to 75% of patients with prior exposure to ruxolitinib, belumosudil, or ibrutinib maintained a response at 12 months post-treatment with axatilimab (0.3 mg/kg Q2W). Surprisingly, the number of patients who maintained a response at 12 months post-treatment with axatilimab (0.3 mg/kg Q2W) was greater for patients who received a prior therapy (ruxolitinib, belumosudil, or ibrutinib) compared to those who did not receive the prior therapy. It was surprisingly shown that greater than or equal to 79.5% of patients who received four or more therapies prior to treatment with axatilimab at the lowest dose (0.3 mg/kg Q2W) maintained a response at 12 months while only 57.7% of patients who received less than four therapies prior to treatment with axatilimab at the lowest dose (0.3 mg/kg Q2W) maintained a response at 12 months. See Table 5 below.

TABLE 5
Efficacy across subgroups treated with axatilimab
at the lowest dose (0.3 mg/kg Q2W)

| | Objective response | Number of |
|---|---|---|
| Subgroup | rate (95% CI) | participants |
| Overall | 73.8 (62.7-83.0) | 80 |

Age group

| | | |
|---|---|---|
| <17 years | 75.0 (19.4-99.4) | 4 |
| ≥17 and <65 years | 78.2 (65.0-88.2) | 55 |
| ≥65 years | 61.9 (38.4-81.9) | 21 |

Number of lines of prior therapy

| | | |
|---|---|---|
| <4 | 57.7 (36.9-76.6) | 26 |
| 4-6 | 79.5 (63.5-90.7) | 39 |
| >6 | 86.7 (59.5-98.3) | 15 |

Prior ibrutinib

| | | |
|---|---|---|
| Yes | 81.5 (61.9-93.7) | 27 |
| No | 69.8 (55.7-81.7) | 53 |

Prior ruxolitinib

| | | |
|---|---|---|
| Yes | 78.9 (66.1-88.6) | 57 |
| No | 60.9 (38.5-80.3) | 23 |

Prior belumosudil

| | | |
|---|---|---|
| Yes | 75.0 (47.6-92.7) | 16 |
| No | 73.4 (60.9-83.7) | 64 |

Severity of cGVHD at screening

| | | |
|---|---|---|
| Mild/Moderate | 64.7 (38.3-85.8) | 17 |
| Severe | 76.2 (63.8-86.0) | 63 |

Number of organs involved at baseline

| | | |
|---|---|---|
| ≤4 | 67.3 (52.9-79.7) | 52 |
| >4 | 85.7 (67.3-96.0) | 28 |

As shown in Table 5, patients with severe cGVHD at screening had a higher objective response rate to treatment with axatilimab (0.3 mg/kg Q2W) than patients with mild or moderate cGVHD at screening. It was surprisingly shown that 76.2% of patients with severe cGVHD at screening maintained a response at 12 months post-treatment with axatilimab (0.3 mg/kg Q2W) while only 64.7% of patients with mild or moderate cGVHD at screening maintained a response at 12 months post-treatment with axatilimab (0.3 mg/kg Q2W). See Table 5.

It was also shown that patients with more severe organ involvement at baseline had a higher objective response rate to treatment with axatilimab (0.3 mg/kg Q2W) than patients with less organ involvement at baseline. For example, 85.7% of patients with >4 organs involved at baseline maintained a response at 12 months post-treatment with axatilimab (0.3 mg/kg Q2W) while only 67.3% of patients with ≤4 organs involved at baseline maintained a response at 12 months post-treatment with axatilimab (0.3 mg/kg Q2W). See Table 5.

For patients treated with axatilimab at the lowest dose (0.3 mg/kg Q2W), responses (complete and partial) were seen in all involved organs, including lungs (15/32; 46.9%), joints/fascia (42/55; 76.4%), and esophagus (18/23; 78.3%). See Table 6 below.

TABLE 6
Organ-specific responses in the lowest dose (0.3 mg/kg Q2W) treatment group

| | Lower GI | Upper GI | Esophagus | Joints/Fascia | Mouth | Lungs | Liver | Eyes | Skin |
|---|---|---|---|---|---|---|---|---|---|
| Parameter | (N = 9) | (N = 11) | (N = 23) | (N = 55) | (N = 40) | (N = 32) | (N = 10) | (N = 59) | (N = 64) |
| RR, % | 88.9 | 81.8 | 78.2 | 76.4 | 52.5 | 46.9 | 40.0 | 30.5 | 26.6 |
| CR, % | 88.9 | 81.8 | 65.2 | 20.0 | 42.5 | 15.6 | 20.0 | 10.2 | 9.4 |
| PR, % | 0.0 | 0.0 | 13.0 | 56.4 | 10.0 | 31.3 | 20.0 | 20.3 | 17.2 |

RR, response rate; CR, complete response; PR, partial response.

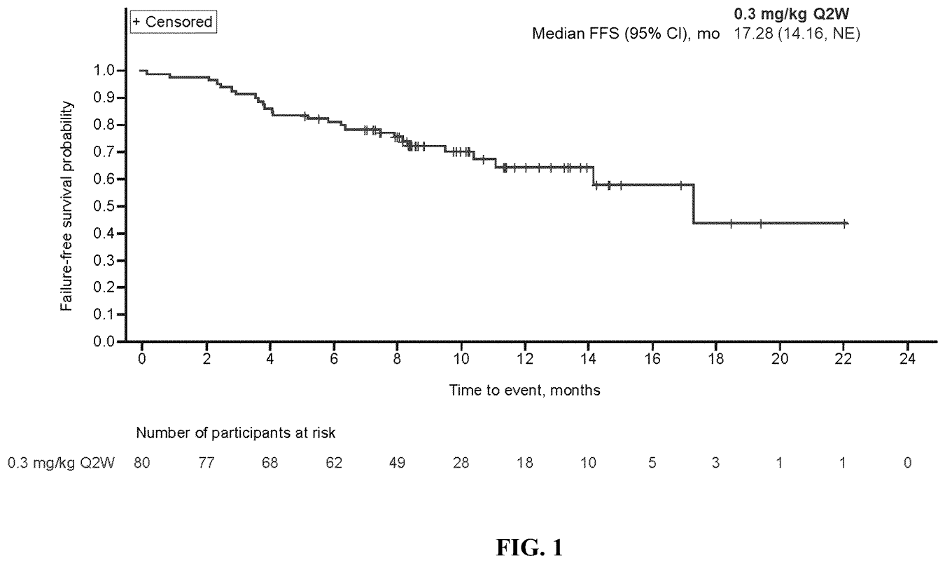

The median failure-free survival (defined as time from randomization to addition of another systemic immune suppressive therapy for cGVHD, relapse of underlying malignancy, or death, whichever is earlier) was 17.3 months (95% CI) for patients treated with axatilimab at the lowest dose (0.3 mg/kg Q2W). See FIG. 1.

mLSS Scores

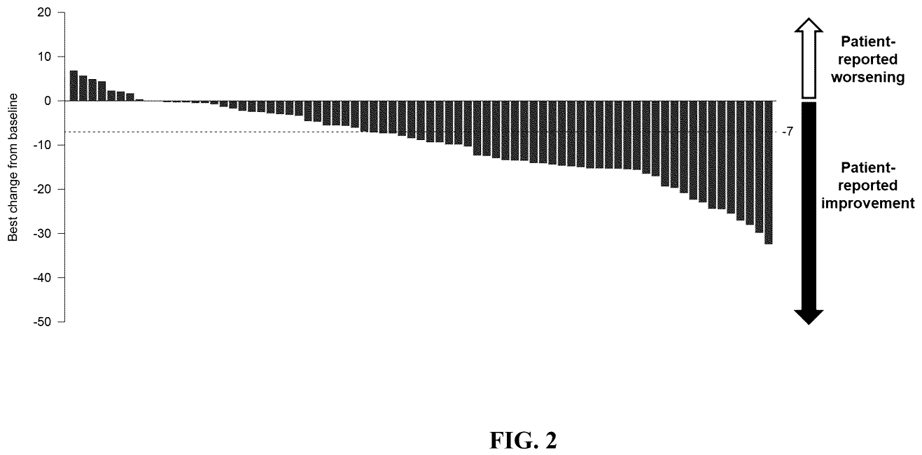

Additionally, 55% of patients treated with axatilimab at the lowest dose (0.3 mg/kg Q2W) experienced a clinically meaningful improvement in symptoms, as measured by at least a seven-point decrease in the mLSS score, while only 36% of patients treated with axatilimab at the highest dose (3.0 mg/kg Q4W) experienced a clinically meaningful improvement in symptoms. The median time to the clinically meaningful improvement in symptoms was 1.5 months for patients treated with axatilimab at the lowest dose (0.3 mg/kg Q2W). FIG. 2 shows the best change of mLSS score from baseline for individual patients treated with axatilimab at a dose of 0.3 mg/kg every two weeks. The number of patients treated with axatilimab at the intermediate dose (1.0 mg/kg Q2W) that experienced a clinically meaningful improvement in symptoms was similar to that of patients treated with axatilimab at the lowest dose (0.3 mg/kg Q2W). For the total patient population (i.e., patients in all three of the treatment groups), about 87% of patients had an improvement in mLSS score from baseline. See Table 7 below.

TABLE 7
mLSS scores per treatment group

| | 0.3 mg/kg | 1 mg/kg | 3 mg/kg |
|---|---|---|---|
| | Q2W | Q2W | Q4W |
| Parameter | (N = 80) | (N = 81) | (N = 80) |
| mLSS response rate | 55 (43, 66) | 54 (42, 65) | 36 (25, 48) |
| within C7D1, % | | | |

### Axatilimab Safety Profile

Axatilimab was also well tolerated by patients treated with the lowest dose (0.3 mg/kg Q2W). The 1 mg/kg Q2W and 3 mg/kg Q4W treatment groups crossed the safety boundary while the 0.3 mg/kg Q2W treatment group did not cross the safety boundary, which was defined as 20% of the patient population experiencing a toxic event per protocol. A toxic event was defined as serious or severe (≥Grade 3) treatment-related treatment emergent adverse events (TEAEs), as well as treatment-related Grade 2 events resulting in medical intervention or hospitalization.

The most common adverse events were consistent with on-target effects of CSF-1R inhibition. There was no incidence of cytomegalovirus or other viral reactivation, and no significant drug-related cytopenias have been reported. For patients treated with axatilimab at 0.3 mg/kg every two weeks, serious or severe (≥Grade 3) treatment-related TEAEs occurred in 14 patients (17.7% of patients), with 5 patients (6.3% of patients) experiencing adverse events leading to discontinuation of treatment. Across treatment groups, serious or severe serious or severe (≥Grade 3) treatment-related TEAEs occurred in 79 patients (33.1% of patients), with a total of 5 patients (15.5% of patients) experiencing adverse events leading to discontinuation of treatment. Dose decreases occurred in 5 patients (6.3% of patients), 6 patients (7.4% of patients), and 13 patients (16.5% of patients) treated with axatilimab at the lowest dose, the intermediate dose, and the highest dose, respectively. See Table 8 below.

TABLE 8
Axatilimab safety profile

| | | | |
|---|---|---|---|
| | 0.3 mg/kg | 1 mg/kg | 3 mg/kg |
| | Q2W | Q2W | Q4W |
| Parameter | (N = 79) | (N = 81) | (N = 79) |

Axatilimab dose changes owning to AE, n (%)

| | | | |
|---|---|---|---|
| Discontinuation | 5 (6.3) | 18 (22.2) | 14 (17.7) |
| Dose decrease | 5 (6.3) | 6 (7.4) | 13 (16.5) |

Any grade AE in ≥20% of total patients

| | | | |
|---|---|---|---|
| Fatigue | 18 (22.8) | 16 (19.8) | 21 (26.6) |
| Headache | 15 (19.0) | 14 (17.3) | 16 (20.3) |
| Periorbital edema | 2 (2.5) | 19 (23.5) | 23 (29.1) |
| COVID-19 | 13 (16.5) | 18 (22.2) | 11 (13.9) |

Laboratory-based abnormalities

| | | | |
|---|---|---|---|
| AST increase | 11 (13.9) | 31 (38.3) | 43 (54.4) |
| CPK increase | 9 (11.4) | 26 (32.1) | 49 (62.0) |
| Lipase increased | 9 (11.4) | 21 (25.9) | 39 (49.4) |
| Lactate dehydrogenase increased | 11 (13.9) | 22 (27.2) | 32 (40.5) |
| ALT increase | 10 (12.7) | 18 (22.2) | 31 (39.2) |
| Amylase increase | 3 (3.8) | 10 (12.3) | 34 (43.0) |
| At least 1 related Grade ≥3 AE, | 14 (17.7) | 28 (34.6) | 37 (46.8) |
| n (%) | | | |
| Fatal AE | 1 (1.3)^{a} | 7 (8.6)^{b} | 6 (7.6)^{c} |

AE, adverse event; ALT, alanine aminotransferase; AST, aspartate aminotransferase; CPK, blood creatine kinase; Q2W, every 2 weeks; Q4W, every 4 weeks.
^{a}Dyspnea.
^{b}Sudden cardiac death, bronchopulmonary aspergillosis, pneumonia (n = 2), sepsis, leukemia recurrent, respiratory failure.
^{c}Leukemia recurrent, dyspnea, respiratory failure, shock hemorrhagic, neutropenic sepsis, pneumonia.

As shown in Table 8, common adverse reactions such as fatigue and headache occurred in similar numbers of patients in each treatment group. However, periorbital edema occurred in significantly less patients treated with axatilimab at the lowest dose compared to patients treated with axatilimab at the intermediate dose or the highest dose. Patients treated with axatilimab at the lowest dose also experienced fewer laboratory-based abnormalities, fewer Grade 3 or greater adverse events, and fewer fatal adverse events as compared to patients treated with axatilimab at the intermediate dose or the highest dose. See Table 8.

Thus, these results demonstrate a durable patient response to treatment with axatilimab at a dose of 0.3 mg/kg every two weeks, with 60% of patients still responding at one year. These results also demonstrate that treatment with axatilimab at a dose of 0.3 mg/kg every two weeks was well tolerated by patients.

### Example 2: Modeling and Simulations to Inform Flat Dosing and Weight-Band Dosing

Simulated steady-state PK/PD parameters (including AUC, Cmax, and Cmin) in adult and pediatric patients for different dosing regimens (weight-based vs. flat vs. weight-band; Q2W vs Q4W) were compared with steady state PK/PD parameters in adult patients who received the 0.3 mg/kg Q2W dose. Scenarios with less than 30% change in exposure as compared to the 0.3 mg/kg Q2W dose in adult patients were simulated to predict efficacy and safety.

The adult virtual population was sampled randomly with replacement from the analysis dataset (i.e., “bootstrap sampling”) for cGVHD subjects older than 18 years of age.

For the pediatric virtual population, age, sex, and corresponding body weights were generated using the model described in Sumpter et al., Paediatr. Anaesth. Predicting weight using postmenstrual age-neonates to adults (2011); 21:309-15. To ensure that the body weight distribution of the virtual pediatric population realistically represented the observed body weight for pediatric patients with cGVHD, individual observed body weight values from studies SNDX 6352 0503 (NCT03604692) and AGAVE 201 (NCT04710576) as well as historical data from Verdú-Amorós et al., Transfusion (Paris) Mini photopheresis for refractory chronic graft-versus-host disease in children and adolescents 2018; 58:2495-500, for patients <18 years old were compared to the median and range of simulated body weight distribution (N=10000) for the corresponding age of each participant. Furthermore, an allometric scaling was applied to the axatilimab linear clearance (CL) and volume of distribution parameters (Vd) such that CL CL_{TV}*(Weight 70)\hat{ }0.75 and Vd_{TV}=Vd*(Weight/70), where CL_{TV}, Vd_{TV} represent typical parameter values.

The choice of weight band limits was based on the quantiles of weight distribution in the respective adult or pediatric populations and reference exposure metrics (i.e., 0.3 mg/kg Q2W in adults). Post-hoc individual parameter estimates were sampled from the final population PK/PD model to account for the variability in steady-state AUC, Cₘₐₓ, and Cₘᵢₙ values. Each simulation was based on 1000 subjects.

The above described simulations were used to identify a flat dosing regimen for adult patients and weight-band dosing regimens for adult and pediatric patients. See Table 9 below.

TABLE 9
Axatilimab dosage regimen in adult and
pediatric patients 6 years and older

| | | |
|---|---|---|
| Body Weight | Adult Patients | Pediatrics Patients |

| | | | | | | |
|---|---|---|---|---|---|---|
| Dosage | <30 | kg | 22 | mg | 9 | mg |
| Regimen 1 | 30-55 | kg | | | 14 | mg |
| | >55 | kg | | | 22 | mg |
| Dosage | <40 | kg | 10.5 | mg | 10.5 | mg |
| Regimen 2 | 40-60 | kg | 17 | mg | 17 | mg |
| | >60 | kg | 23 | mg | 23 | mg |

The above described simulations were also used to identify a weight-based dose of 0.35 mg/kg for pediatric patients, assuming a median weight in a pediatric patient population of 42.2 kg with a range of 14.2 kg to 106 kg.

These doses and dosing regimens were predicted to have comparable pharmacokinetic exposure (for example, a change of less than 30%) and pharmacodynamics (for example, comparable levels of circulating non-classical monocytes), and similar efficacy (for example, a change of less than 2%) and safety (for example, a change of less than 1%) as compared to the 0.3 mg/kg Q2W dose in adult patients.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-58. (canceled)

59. A method of treating chronic graft-versus-host disease (cGVHD) in a human subject in need thereof, the method comprising administering to the human subject a therapeutically effective amount of an antibody that binds to colony stimulating factor 1 receptor (CSF-1R), wherein the antibody comprises a variable heavy (VH) domain comprising VH complementarity determining region (CDR)1 (VH CDR1), VH CDR2, and VH CDR3,
wherein:
the VH CDR1 comprises the amino acid sequence GFSLTTYGMGVG (SEQ ID NO:6);
the VH CDR2 comprises the amino acid sequence NIWWDDDKYYNPSLKN (SEQ ID NO:7); and
the VH CDR3 comprises the amino acid sequence IGPIKYPTAPYRYFDF (SEQ ID NO:8); and
wherein the antibody comprises a variable light (VL) domain comprising VL CDR1, VL CDR2, and VL CDR3, wherein:
the VL CDR1 comprises the amino acid sequence LASEDIYDNLA (SEQ ID NO:9);
the VL CDR2 comprises the amino acid sequence YASSLQD (SEQ ID NO:10); and
the VL CDR3 comprises the amino acid sequence LQDSEYPWT (SEQ ID NO:11), and
wherein:
(i) the human subject is an adult patient or a pediatric patient with a body weight greater than about 55 kg and the antibody is administered intravenously once every two weeks at a dose of about 22 mg;
(ii) the human subject is a pediatric patient with a body weight greater than or equal to about 30 kg and less than or equal to about 55 kg and the antibody is administered intravenously once every two weeks at a dose of about 14 mg; or
(iii) the human subject is a pediatric patient with a body weight less than about 30 kg and the antibody is administered intravenously once every two weeks at a dose of about 9 mg.

60. The method of claim 59, wherein the antibody is administered after failure of at least two prior lines of systemic therapy.

61. The method of claim 59, wherein the cGVHD is recurrent or refractory active cGVHD.

62. The method of claim 59, wherein the human subject was previously treated with ibrutinib or ruxolitinib.

63. The method of claim 59, wherein the cGVHD is severe cGVHD.

64. The method of claim 59, wherein the human subject exhibited manifestations of cGVHD in greater than or equal to four organs at baseline.

65. The method of claim 59, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5).

66. The method of claim 59, wherein the antibody comprises a heavy chain and a light chain, and wherein the heavy chain comprises the amino acid sequence set forth in SEQ ID NO:12 and the light chain comprises the amino acid sequence set forth in SEQ ID NO:3.

67. A method of treating chronic graft-versus-host disease (cGVHD) in a human subject in need thereof, the method comprising administering to the human subject a therapeutically effective amount of an antibody that binds to colony stimulating factor 1 receptor (CSF-1R), wherein the antibody comprises a variable heavy (VH) domain comprising VH complementarity determining region (CDR)1 (VH CDR1), VH CDR2, and VH CDR3,
wherein:
the VH CDR1 comprises the amino acid sequence GFSLTTYGMGVG (SEQ ID NO:6);
the VH CDR2 comprises the amino acid sequence NIWWDDDKYYNPSLKN (SEQ ID NO:7); and
the VH CDR3 comprises the amino acid sequence IGPIKYPTAPYRYFDF (SEQ ID NO:8); and
wherein the antibody comprises a variable light (VL) domain comprising VL CDR1, VL CDR2, and VL CDR3, wherein:
the VL CDR1 comprises the amino acid sequence LASEDIYDNLA (SEQ ID NO:9);
the VL CDR2 comprises the amino acid sequence YASSLQD (SEQ ID NO:10); and
the VL CDR3 comprises the amino acid sequence LQDSEYPWT (SEQ ID NO:11), and
wherein:
(i) the human subject has a body weight greater than about 60 kg and the antibody is administered intravenously once every two weeks at a dose of about 23 mg;
(ii) the human subject has a body weight greater than or equal to about 40 kg and less than or equal to about 60 kg and the antibody is administered intravenously once every two weeks at a dose of about 17 mg; or
(iii) the human subject has a body weight less than about 40 kg and the antibody is administered intravenously once every two weeks at a dose of about 10.5 mg.

68. The method of claim 67, wherein the antibody is administered after failure of at least two prior lines of systemic therapy.

69. The method of claim 67, wherein the cGVHD is recurrent or refractory active cGVHD.

70. The method of claim 67, wherein the human subject was prior treated with ibrutinib or ruxolitinib.

71. The method of claim 67, wherein the cGVHD is severe cGVHD.

72. The method of claim 67, wherein the human subject exhibited manifestations of cGVHD in greater than or equal to four organs at baseline.

73. The method of claim 67, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5).

74. The method of claim 67, wherein the antibody comprises a heavy chain and a light chain, and wherein the heavy chain comprises the amino acid sequence set forth in SEQ ID NO:12 and the light chain comprises the amino acid sequence set forth in SEQ ID NO:3.

75. A method of treating chronic graft-versus-host disease (cGVHD) in a pediatric human subject in need thereof, the method comprising administering to the pediatric human subject a therapeutically effective amount of an antibody that binds to colony stimulating factor 1 receptor (CSF-1R), wherein the antibody comprises a variable heavy (VH) domain comprising VH complementarity determining region (CDR)1 (VH CDR1), VH CDR2, and VH CDR3,
wherein:
the VH CDR1 comprises the amino acid sequence GFSLTTYGMGVG (SEQ ID NO:6);
the VH CDR2 comprises the amino acid sequence NIWWDDDKYYNPSLKN (SEQ ID NO:7); and
the VH CDR3 comprises the amino acid sequence IGPIKYPTAPYRYFDF (SEQ ID NO:8); and
wherein the antibody comprises a variable light (VL) domain comprising VL CDR1, VL CDR2, and VL CDR3, wherein:
the VL CDR1 comprises the amino acid sequence LASEDIYDNLA (SEQ ID NO:9);
the VL CDR2 comprises the amino acid sequence YASSLQD (SEQ ID NO:10); and
the VL CDR3 comprises the amino acid sequence LQDSEYPWT (SEQ ID NO:11), and
wherein the antibody is administered intravenously once every two weeks at a dose of 0.35 mg/kg body weight of the treated pediatric human subject.

76. The method of claim 75, wherein the cGVHD is recurrent or refractory active cGVHD.

77. The method of claim 75, wherein the human subject was prior treated with ibrutinib or ruxolitinib.

78. The method of claim 75, wherein the cGVHD is severe cGVHD.

79. The method of claim 75, wherein the human subject exhibited manifestations of cGVHD in greater than or equal to four organs at baseline.

80. A method of treating chronic graft-versus-host disease (cGVHD) in a population of human subjects in need thereof, the method comprising administering to each member of the population of human subjects a therapeutically effective amount of an antibody that binds to colony stimulating factor 1 receptor (CSF-1R), wherein the antibody comprises a variable heavy (VH) domain comprising VH complementarity determining region (CDR)1 (VH CDR1), VH CDR2, and VH CDR3,
wherein:
the VH CDR1 comprises the amino acid sequence GFSLTTYGMGVG (SEQ ID NO:6);
the VH CDR2 comprises the amino acid sequence NIWWDDDKYYNPSLKN (SEQ ID NO:7); and
the VH CDR3 comprises the amino acid sequence IGPIKYPTAPYRYFDF (SEQ ID NO:8); and
wherein the antibody comprises a variable light (VL) domain comprising VL CDR1, VL CDR2, and VL CDR3, wherein:
the VL CDR1 comprises the amino acid sequence LASEDIYDNLA (SEQ ID NO:9);
the VL CDR2 comprises the amino acid sequence YASSLQD (SEQ ID NO:10); and
the VL CDR3 comprises the amino acid sequence LQDSEYPWT (SEQ ID NO:11),
wherein the antibody is administered intravenously once every two weeks at a dose of 0.3 mg/kg body weight of each treated human subject, and
wherein the overall response rate in the population of human subjects within the first six months after initiation of treatment is at least 60%.

81. The method of claim 80, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5).

82. The method of claim 80, wherein the antibody comprises a heavy chain and a light chain, and wherein the heavy chain comprises the amino acid sequence set forth in SEQ ID NO:12 and the light chain comprises the amino acid sequence set forth in SEQ ID NO:3.

83. The method of claim 80, wherein the cGVHD is recurrent or refractory active cGVHD.

84. The method of claim 80, wherein the cGVHD is recurrent or refractory active cGVHD after two or more lines of systemic therapy.

85. The method of claim 80, wherein:
(i) the overall response rate in the population of human subjects within the first six months after initiation of treatment is at least 65%, at least 70%, or at least 74%,
(ii) the duration of response sensitivity in the population of human subjects is at least 50%, at least 55%, or at least 60%, and/or
(iii) the modified Lee Symptom Scale (mLSS) response rate in the population of human subjects is at least 45%, at least 50%, or at least 55%.

86. The method of claim 80, wherein the antibody that binds to colony stimulating factor 1 receptor (CSF-1R) is axatilimab.


SEQUENCE LISTING (12 sequences)

SEQ ID NO: 1 - protein, 979 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFCSEQIDNO

SEQ ID NO: 2 - protein, 460 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSEQIDNO

SEQ ID NO: 3 - protein, 221 aa, synthetic construct
DIQMTQSPSSLSASVGDRVTITCLASEDIYDNLAWYQQKPGKAPKLLIYYASSLQDGVPSRFSGSGSGTDYTLTISSLQPEDFATYYCLQDSEYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSEQIDNO

SEQ ID NO: 4 - protein, 132 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSEQIDNO

SEQ ID NO: 5 - protein, 114 aa, synthetic construct
DIQMTQSPSSLSASVGDRVTITCLASEDIYDNLAWYQQKPGKAPKLLIYYASSLQDGVPSRFSGSGSGTDYTLTISSLQPEDFATYYCLQDSEYPWTFGGGTKVEIKSEQIDNO

SEQ ID NO: 6 - protein, 12 aa, synthetic construct
GFSLTTYGMGVG

SEQ ID NO: 7 - protein, 16 aa, synthetic construct
NIWWDDDKYYNPSLKN

SEQ ID NO: 8 - protein, 16 aa, synthetic construct
IGPIKYPTAPYRYFDF

SEQ ID NO: 9 - protein, 11 aa, synthetic construct
LASEDIYDNLA

SEQ ID NO: 10 - protein, 7 aa, synthetic construct
YASSLQD

SEQ ID NO: 11 - protein, 9 aa, synthetic construct
LQDSEYPWT

SEQ ID NO: 12 - protein, 459 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSEQIDNO